(19) United States
(12) Patent Application Publication
(10) Pub. No.: US 2019/0183960 A1
(43) Pub. Date: Jun. 20, 2019

(54) METHOD TO TREAT ASTHMA AND AIRWAY ALLERGY USING CYCLO TYROSINE-VALINE AND RELATED COMPOUNDS

(71) Applicant: Chang Gung Biotechnology Corp., Taipei (TW)

(72) Inventors: Yun-Fei Ko, Taipei (TW); Jan Martel, Taoyuan (TW); Jian-Ching Liau, Taipei (TW); Chen-Yaw Chiu, New Taipei City (TW); Chuan-Sheng Lin, Taoyuan (TW); Luis Dominick B. Antig, Taoyuan (TW); Pei-Wen Hsieh, Taoyuan (TW); Yu-Li Chen, Taoyuan (TW); Chih-Jung Chang, Taoyuan (TW); Tsung-Ru Wu, Taoyuan (TW); David Marcelo Ojcius, San Francisco, CA (US); Chia-Chen Lu, New Taipei City (TW); Hsin-Chih Lai, Taoyuan (TW); John D. Young, Taipei (TW)

(21) Appl. No.: 15/906,353

(22) Filed: Feb. 27, 2018

(30) Foreign Application Priority Data
Dec. 15, 2017 (TW) 106144234

(51) Int. Cl. A61K 38/05; A61P 11/06

(52) U.S. Cl. CPC A61K 38/05; A61P 11/06

(57) ABSTRACT

The present invention provides a method to treat asthma and airway allergy by administering the dipeptide cyclo tyrosine-valine or a related cyclodipeptide compound to a subject in need thereof. Cyclo tyrosine-valine or the related cyclodipeptide compound reduces Th2 cell activation, airway hyperresponsiveness, accumulation of immune cells in bronchoalveolar fluid, and allergen-specific antibody production in an animal or human subject. The compound can therefore be used to treat asthma and airway allergy.

### CROSS-REFERENCE TO RELATED APPLICATION

This application claims priority of Taiwan patent application No. 106144234, filed on Dec. 15, 2017, the content of which is incorporated herein in its entirety by reference.

### BACKGROUND OF THE INVENTION

### 1. Field of the Invention

The present invention relates to a method to treat asthma using a cyclodipeptide compound. Particularly, the present invention provides a method to treat asthma and airway allergy using cyclo tyrosine-valine or a related cyclodipeptide compound.

### 2. The Prior Art

Asthma is a common disease condition in which inflammation develops in the airway and the lungs. It is estimated that between 235 and 330 million people suffer from asthma worldwide, leading to 250,000-345,000 deaths annually. Notably, asthma is more prevalent in developed countries, including the United Kingdom, New Zealand, the United States of America, Canada and Australia. Asthmatic patients often experience symptoms such as cough, wheezing, and shortness of breath, which considerably affect quality of life and may lead to complications and death.

Asthma is characterized by accumulation and activation of immune cells in the airway mucosa, including eosinophils, neutrophils, macrophages, and T cells, which secrete a variety of immunological mediators. During the process of airway inflammation, naive CD4⁺ T helper cells differentiate into type 2 helper T cells (Th2 cells), and secrete pro-inflammatory cytokines, such as interleukin (IL)-4, IL-5 and IL-13, which induce antibody production and eosinophil activation, further contributing to inflammation in the airway. While no cure exists for asthma, pharmaceutical drugs such as corticosteroids, bronchodilators, β2-adrenergic receptor agonists, anti-cholinergic drugs, and anti-leukotriene agents are usually used to reduce symptoms. Nevertheless, these drugs produce serious side effects and only provide temporary symptom relief.

Asthma may be considered an allergic reaction of the airway when symptoms occur in response to allergens such as hay or pollen. Allergies represent hypersensitive reactions of the immune system against substances found in the environment, including food, hay, pollen, insect stings, or pharmaceutical drugs. Acute allergies may lead to a serious condition called anaphylactic shock which may cause death. Allergies are characterized by binding of immunoglobulin E (IgE) antibodies to an allergen, followed by activation of basophils, secretion of histamine and other pro-inflammatory mediators, and induction of the inflammatory response. Prevention of airway allergies usually requires that the individual avoids allergens, air pollution or cigarette smoking. Although many pharmaceutical drugs are available to treat symptoms of pulmonary allergies, for example, anti-histamines, epinephrine, glucocorticoids, and anti-leukotriene agents, these drugs only reduce the severity of symptoms and may produce adverse side effects.

In view of the abovementioned drawbacks of common drugs for asthma and airway allergies, it is of great necessity to develop novel agents for treating these conditions.

### SUMMARY OF THE INVENTION

The present invention provides a method for treating asthma and airway allergy, comprising administering to a subject in need thereof an effective amount of cyclo tyrosine-valine or a related cyclodipeptide compound.

In one embodiment of the present invention, the effective amount of cyclo tyrosine-valine or the related cyclodipeptide compound is from 0.001 mg/kg to 1,000 mg/kg of body weight per day. In one preferred embodiment, the effective amount of the compound is 0.003 mg/kg of body weight per day.

In one embodiment of the present invention, the cyclo tyrosine-valine or the related cyclodipeptide compound is administered via intraperitoneal injection, inhalation, or by mouth.

In one embodiment of the present invention, the related cyclodipeptide compound is cyclo tyrosine-leucine, which has an isobutyl group substituted for the isopropyl group of valine.

In one embodiment of the present invention, cyclo tyrosine-valine or the related cyclodipeptide compound significantly reduces secretion of pro-inflammatory cytokines, airway hyperresponsiveness (AHR), eosinophil accumulation in the lungs, and production of allergen-specific antibodies. The pro-inflammatory cytokines include interleukin-4 (IL-4), interleukin-5 (IL-5), interleukin-13 (IL-13), or any combinations thereof; the allergen-specific antibody is an immunoglobulin E (IgE), an immunoglobulin G (IgG), or combinations thereof.

The present invention discloses that cyclo tyrosine-valine or the related cyclodipeptide compound significantly reduces the symptoms of asthma and airway allergies in mammals, without producing toxic side effects. Accordingly, the present invention provides a new strategy to treat asthma and airway allergies that utilizes cyclo tyrosine-valine or a related cyclodipeptide compound.

The present invention is further explained in the following drawings and examples. It is understood that the examples given below do not, however, limit the scope of the invention, and it will be evident to those skilled in the art that modifications can be made without departing from the scope of the appended claims.

### BRIEF DESCRIPTION OF THE DRAWINGS

FIG. 1 shows the inhibitory effect of cyclo tyrosine-valine (hereafter referred to as cyclo Tyr-Val) on the secretion of the pro-inflammatory cytokine IL-4 by Th2 cells derived from BALB/c mice; * and **** indicate P<0.05 and P<0.0001, respectively, compared with the vehicle-treated group receiving 0.5% dimethyl sulfoxide (DMSO).

FIG. 2 shows the absence of cytotoxic effects produced by various concentrations of cyclo Tyr-Val on Th2 cells derived from BALB/c mice; “ns” indicates that the results are statistically non-significant.

FIG. 3A shows the dose-dependent inhibitory effect of cyclo Tyr-Val on IL-4 secretion by Th2 cells derived from BALB/c mice; * and **** indicate P<0.05 and P<0.0001, respectively, compared with the vehicle-treated group (0.5% DMSO).

FIG. 3B shows the dose-dependent inhibitory effect of cyclo Tyr-Val on IL-4 secretion by Th2 cells derived from C57BL/6J mice; * and **** indicate P<0.05 and P<0.0001, respectively, compared with the vehicle-treated group (0.5% DMSO).

FIG. 3C shows relative data of the inhibitory effect of cyclo Tyr-Val on IL-4 secretion by Th2 cells derived from BALB/c mice; the data are derived from FIG. 3A.

FIG. 3D shows relative data of the inhibitory effect of cyclo Tyr-Val on IL-4 secretion by Th2 cells derived from C57BL/6J mice; the data are derived from FIG. 3B.

FIG. 4A shows the inhibitory effect of cyclo Tyr-Val on the secretion of pro-inflammatory IL-4 by Th2 cells derived from BALB/c mice; **** indicates P<0.0001 based on unpaired Student's t test compared with the vehicle-treated group (0.5% DMSO).

FIG. 4B shows the inhibitory effect of cyclo Tyr-Val on the secretion of the pro-inflammatory cytokine IL-5 by Th2 cells derived from BALB/c mice; ** indicates P<0.01 based on unpaired Student's t test compared with the vehicle-treated group (0.5% DMSO).

FIG. 4C shows the inhibitory effect of cyclo Tyr-Val on the secretion of the pro-inflammatory cytokine IL-13 by Th2 cells derived from BALB/c mice; *** indicates P<0.001 based on unpaired Student's t test compared with the vehicle-treated group (0.5% DMSO).

FIG. 5 shows the dose-dependent inhibitory effect of cyclo Tyr-Val on ovalbumin (OVA)-induced airway hyperresponsiveness (AHR) in BALB/c mice; * and **** indicate P<0.05 and P<0.0001, respectively, compared with the vehicle-treated group (0.5% DMSO).

FIG. 6A shows that cyclo Tyr-Val dose-dependently reduces OVA-induced cell infiltration in the bronchoalveolar lavage fluid (BALF) of BALB/c mice; **** indicates P<0.0001 compared with the vehicle-treated group (0.5% DMSO).

FIG. 6B shows that cyclo Tyr-Val dose-dependently reduces OVA-induced eosinophilia in the BALF of BALB/c mice; * and **** indicate P<0.05 and P<0.0001, respectively, compared with the vehicle-treated group (0.5% DMSO).

FIG. 7A shows that cyclo Tyr-Val dose-dependently reduces OVA-induced IL-4 production in the BALF of BALB/c mice; *** and **** indicate P<0.001 and P<0.0001, respectively, compared with the vehicle-treated group (0.5% DMSO).

FIG. 7B shows that cyclo Tyr-Val dose-dependently reduces OVA-induced IL-5 production in the BALF of BALB/c mice; **** indicates P<0.0001, respectively, compared with the vehicle-treated group (0.5% DMSO).

FIG. 7C shows that cyclo Tyr-Val dose-dependently reduces OVA-induced IL-13 production in the BALF of BALB/c mice; *** and **** indicate P<0.001 and P<0.0001, respectively, compared with the vehicle-treated group (0.5% DMSO).

FIG. 8A shows that cyclo Tyr-Val dose-dependently reduces OVA-specific IgE production in the sera of OVA-treated BALB/c mice; ** and **** indicate P<0.01 and P<0.0001, respectively, compared with the vehicle-treated group (0.5% DMSO).

FIG. 8B shows that cyclo Tyr-Val dose-dependently reduces OVA-specific IgG1 production in the sera of OVA-treated BALB/c mice; * and **** indicate P<0.05 and P<0.0001, respectively, compared with the vehicle-treated group (0.5% DMSO).

FIG. 8C shows that cyclo Tyr-Val dose-dependently reduces OVA-specific IgG2a production in the sera of OVA-treated BALB/c mice; **** indicates P<0.0001 compared with the vehicle-treated group (0.5% DMSO).

FIG. 9A shows the effects of various modes of administration of cyclo Tyr-Val on OVA-induced AHR in BALB/c mice; **** indicates P<0.0001 based on two-way ANOVA and Bonferroni post hoc test compared with the vehicle-treated group (0.5% DMSO); IP, intraperitoneal injection; IH, inhalation.

FIG. 9B shows the effects of various modes of administration of cyclo Tyr-Val on OVA-induced cell infiltration in the BALF of BALB/c mice; **** indicates P<0.0001 compared with the vehicle-treated group (0.5% DMSO).

FIG. 9C shows the effects of various modes of administration of cyclo Tyr-Val on OVA-induced eosinophilia in the BALF of BALB/c mice; **** indicates P<0.0001 compared with the vehicle-treated group (0.5% DMSO).

FIG. 10A shows that cyclo Tyr-Val inhibits AHR in BALB/c mice after establishment of OVA-induced AHR; **** indicates P<0.0001 based on two-way ANOVA and Bonferroni post hoc test compared with the vehicle-treated group (0.5% DMSO).

FIG. 10B shows that cyclo Tyr-Val reduces cell infiltration in the BALF of BALB/c mice after establishment of OVA-induced AHR; **** indicates P<0.0001 compared with the vehicle-treated group (0.5% DMSO).

FIG. 10C shows that cyclo Tyr-Val reduces eosinophilia in the BALF of BALB/c mice after establishment of OVA-induced AHR; **** indicates P<0.0001 compared with the vehicle-treated group (0.5% DMSO).

### DETAILED DESCRIPTION OF THE PREFERRED EMBODIMENT

### Definition

The expression “effective amount” or “effective dosage” used herein refers to the amount of cyclo tyrosine-valine that can reduce pro-inflammatory cytokine secretion, cell infiltration in bronchoalveolar lavage fluid, and airway hyperresponsiveness in animals and humans. The effective amount may vary depending on the organism or individual treated but can be determined experimentally using various techniques, including a dose escalation study.

The expression “related cyclodipeptide compound” refers to a compound obtained from cyclo tyrosine-valine in which the functional groups have been chemically modified; these functional groups include the hydrogen, hydroxyl group, isopropyl group, carbonyl group and the nitrogen atom. The related cyclodipeptide compound exhibits anti-asthmatic and anti-allergic activities similar to those produced by cyclo tyrosine-valine.

The data provided in the specification represent approximated, experimental values that may vary within a range of ±20%, preferably ±10%, and most preferably ±5%.

The present invention provides a method to treat asthma and airway allergy, including administering to a subject in need an effective amount of cyclo tyrosine-valine or a related cyclodipeptide compound. The experiments described below show that administration of an effective amount of cyclo tyrosine-valine via various routes completely suppresses or significantly reduces the development and symptoms of asthma and airway allergies in an asthmatic subject. Generally, cyclo tyrosine-valine can be given to mammals and humans at a dose of 0.001-1,000 mg/kg of body weight per day. Details of the present invention are provided below.

### Materials and Methods

### Cyclo Tyrosine-Valine

Cyclo tyrosine-valine used in the examples may be purchased from chemical manufacturers or purified from microorganisms, such as the halophilic bacterium Nocardiopsis gilva. The cyclo tyrosine-valine has the following chemical structure:

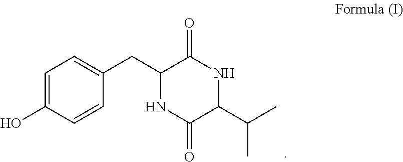 [SMILES: CC(C)C1NC(=O)C(Cc2ccc(O)cc2)NC1=O]

### Statistical Analysis

Data are presented as means±standard deviation (each group consists of 8 mice). Unless indicated otherwise, the data were analyzed using one-way ANOVA followed by Bonferroni post hoc test.

### Example 1

### Cyclo Tyr-Val Inhibits Pro-Inflammatory Cytokine Secretion by Th2 Cells

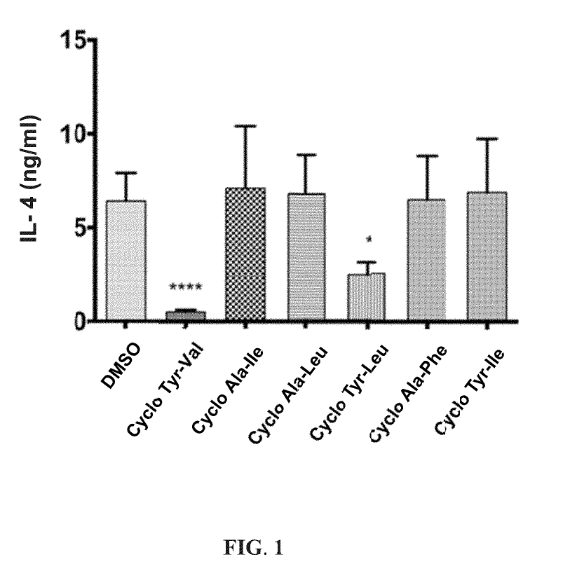

Given that Th2 cells mediate the immunological effects of asthma in humans and animals, we tested the effects of cyclo Tyr-Val on Th2 cells in culture. Naive T cells were isolated from BALB/c mice, and the cells were incubated in a culture medium containing anti-CD3 antibody (2 μg/ml), anti-CD28 antibody (2 μg/ml), anti-interferon-γ (IFN-γ) antibody (10 μg/ml), and IL-4 (10 ng/ml) in order to induce T cell differentiation into Th2 cells. The Th2 cells were treated for 3 days with 2.3 μM of cyclo Tyr-Val, cyclo alanine-isoleucine (cyclo Ala-Ile), cyclo alanine-leucine (cyclo Ala-Leu), cyclo tyrosine-leucine (cyclo Tyr-Leu), cyclo alanine-phenylalanine (cyclo Ala-Phe), or cyclo tyrosine-isoleucine (cyclo Tyr-Ile), and IL-4 protein level was measured using an enzyme-linked immunosorbent assay (ELISA) kit (eBioscience, USA). As shown in FIG. 1, cyclo Tyr-Val significantly inhibited the secretion of pro-inflammatory IL-4 by Th2 cells compared to the control cells treated with the vehicle, which consisted of 0.5% DMSO diluted in sterile saline. Notably, the other cyclic dipeptides tested failed to inhibit IL-4 secretion, with the exception of cyclo Tyr-Leu which inhibited IL-4 to some extent (FIG. 1).

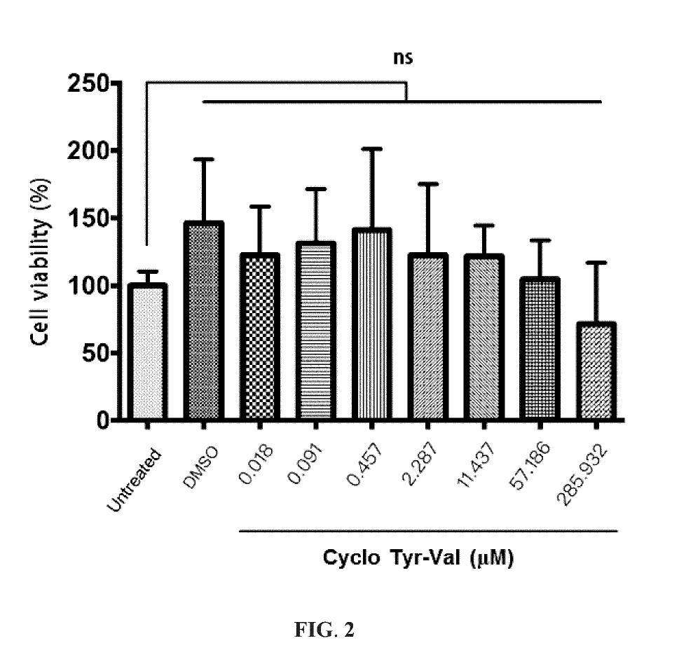

Experiments were performed to examine the effects of cyclo Tyr-Val on cell viability. Th2 cells isolated from BALB/c mice were either left untreated or treated for 3 days with the vehicle (0.5% DMSO in saline) or cyclo Tyr-Val at a concentration of 0.018, 0.091, 0.457, 2.287, 11.438, 57.186, or 285.932 μM. The viability of these cells was assessed using the WST-1 kit (Roche, Switzerland) according to the manufacturer's instructions. As shown in FIG. 2, cyclo Tyr-Val used at the indicated concentrations produced no cytotoxic effects on cultured Th2 cells.

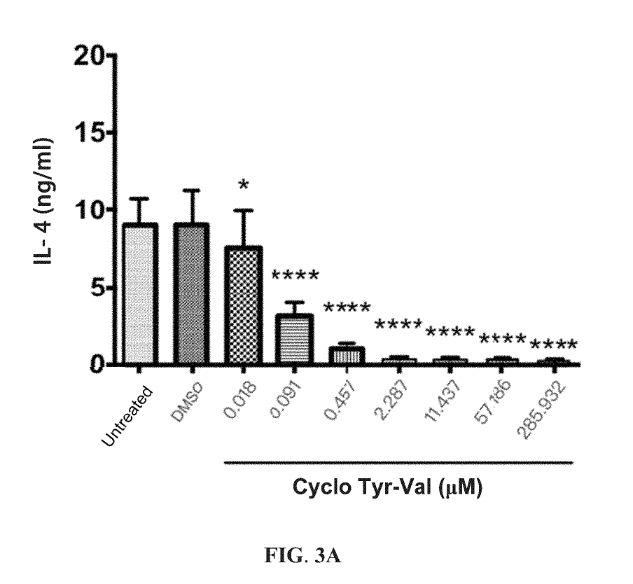

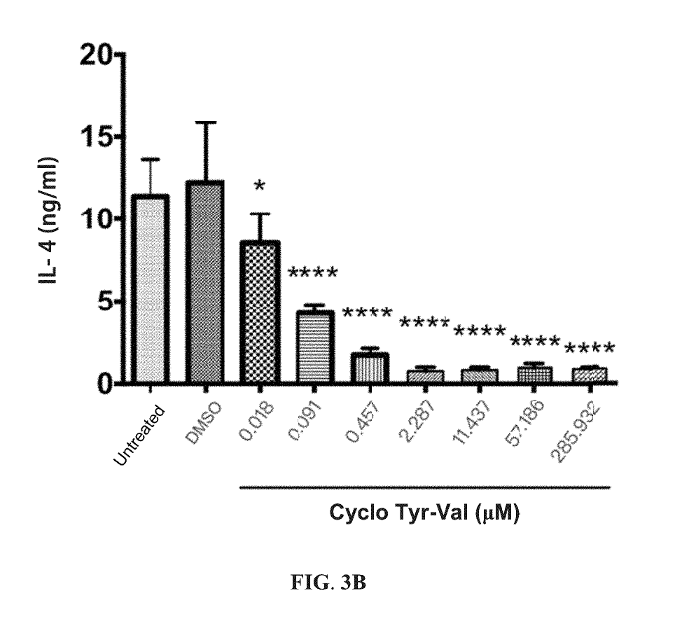

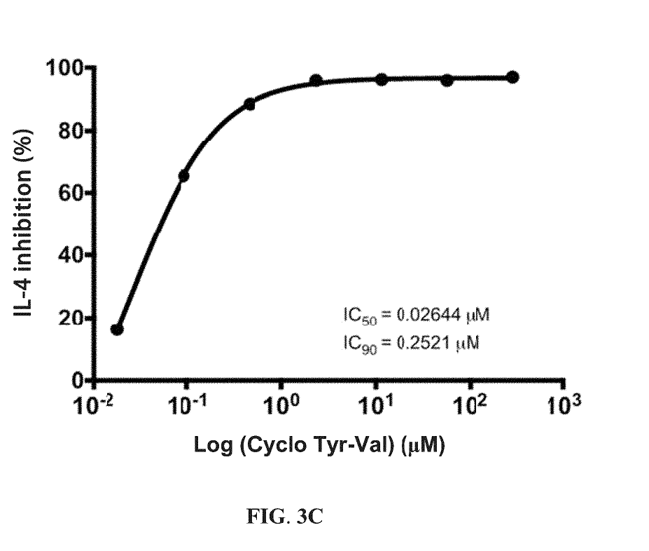

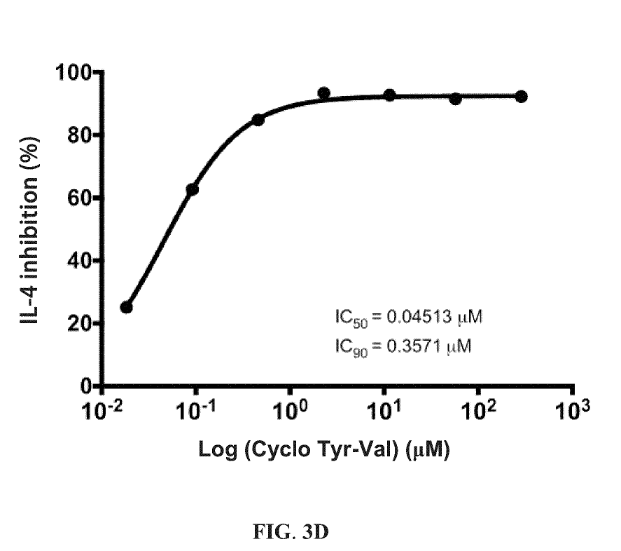

We confirmed that cyclo Tyr-Val inhibits IL-4 secretion by Th2 cells. Th2 cells isolated from BALB/c or C57BL/6J mice were either left untreated or treated for 3 days with the vehicle (0.5% DMSO in saline) or various concentrations of cyclo Tyr-Val, followed by detection of IL-4 production using ELISA. As shown in FIGS. 3A-3D, cyclo Tyr-Val inhibited IL-4 secretion in a dose-dependent manner in Th2 cells from the two strains of mice. FIG. 3C shows that the concentrations of cyclo Tyr-Val required to inhibit IL-4 secretion by 50% (IC₅₀) and 90% (IC₉₀) in Th2 cells isolated from BALB/c mice are 0.02644 μM and 0.2521 μM, respectively; FIG. 3D indicates that the IC₅₀ and IC₉₀ concentrations of cyclo Tyr-Val required to inhibit IL-4 secretion in Th2 cells isolated from C57BL/6J mice are 0.04513 μM and 0.3571 μM, respectively.

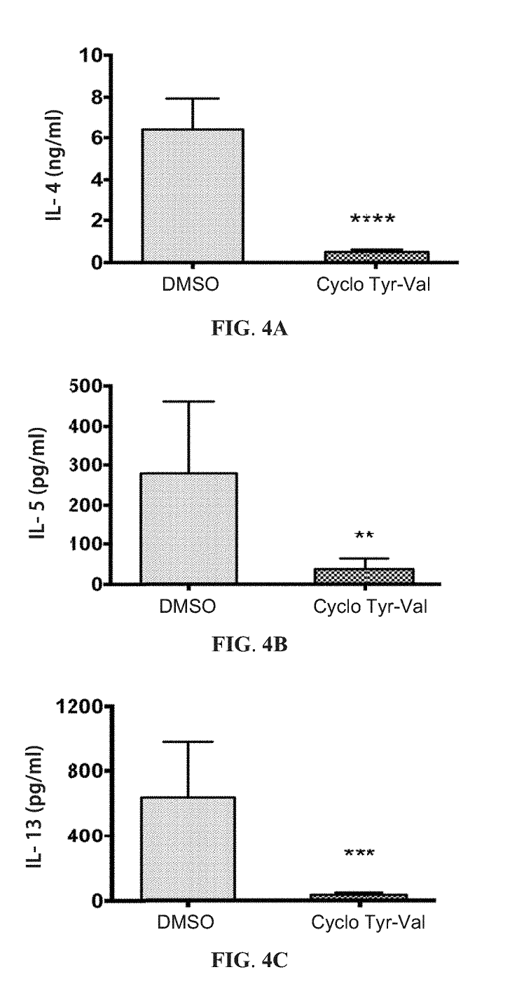

The effects of cyclo Tyr-Val on the secretion of other pro-inflammatory cytokines were investigated. Th2 cells isolated from BALB/c mice were treated for 3 days with the vehicle (0.5% DMSO in saline) or cyclo Tyr-Val at 0.25 μM (corresponding to the IC₉₀ value determined in FIG. 3C), followed by detection of IL-4, IL-5, and IL-13 production using ELISA (eBioscience). As shown in FIGS. 4A-4C, cyclo Tyr-Val inhibited not only the secretion of IL-4, but also that of IL-5 and IL-13. These results show that cyclo Tyr-Val reduces the pro-inflammatory activities of Th2 cells in culture.

### Example 2

### Cyclo Tyr-Val Inhibits Airway Hyperresponsiveness (AHR) in Mice

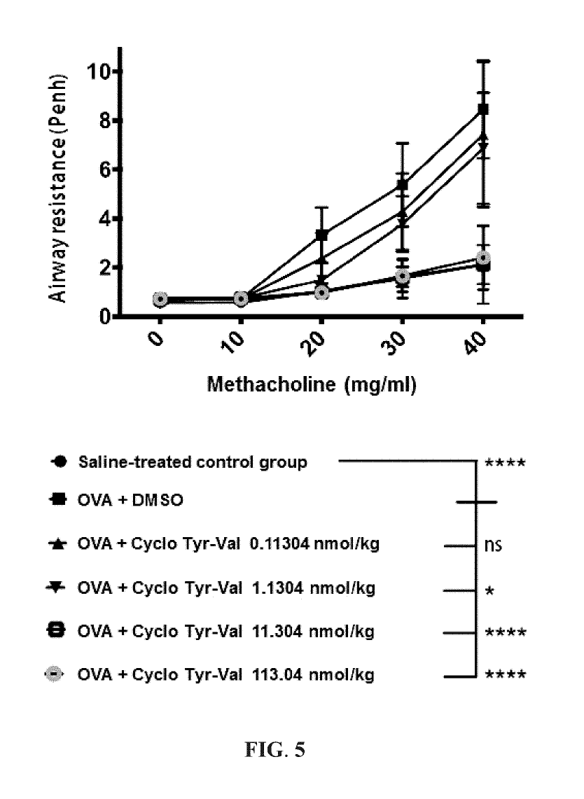

Given that asthma is characterized by hyperresponsiveness to airway stimulants, we examined the effect of cyclo Tyr-Val on AHR in the OVA-induced asthmatic mice stimulated with the exemplary antigen OVA and methacholine (Mch). BALB/c mice were treated intraperitoneally with 50 μg of OVA (Sigma, USA) mixed with 0.8 mg of aluminum hydroxide (Thermo Fischer Scientific, USA) on day 0, 1, 2, and 14, and treated with 2% OVA for 20 minutes using a nebulizer (Pulmo-Aide 5650D; DeVilbiss, USA) on day 14, 17, 21, 24, and 27. The OVA-induced asthmatic mice were intraperitoneally treated with the vehicle (0.5% DMSO in saline) or cyclo Tyr-Val at a dose of 0.11304, 1.1304, 11.304, or 113.04 nmol/kg of body weight per day on day 21 to 27. Airway resistance (Penh) was measured using a whole-body plethysmography (WBP) chamber (Buxco Electronics, USA) after stimulation with methacholine at doses of 0, 10, 20, 30, and 40 mg/ml. As shown in FIG. 5, cyclo Tyr-Val inhibited OVA-induced AHR in a dose-dependent manner following stimulation with methacholine.

### Example 3

### Cyclo Tyr-Val Inhibits Cell Infiltration in the Lungs, Pro-Inflammatory Cytokine Secretion, and Antibody Production in Mice

Given that asthma is characterized by accumulation of immune cells including eosinophils in lung tissues, we studied the effects of cyclo Tyr-Val on BALF cell count, pro-inflammatory cytokine secretion, and antibody production. OVA-induced asthmatic mice (BALB/c) were established according to the method described in Example 2. After the mice were intraperitoneally treated with the vehicle (0.5% DMSO in saline) or cyclo Tyr-Val at a dose of 0.11304, 1.1304, 11.304, or 113.04 nmol/kg of body weight per day on day 21 to 27, BALF and serum were collected. Total cell count in BALF was determined using a hemocytometer; eosinophils were counted after isolation using Cytospin. Protein levels of IL-4, IL-5, and IL-13 in BALF were determined using ELISA. Serum protein levels of OVA-specific antibodies, including IgE, IgG1, and IgG2a, were determined using ELISA (eBioscience).

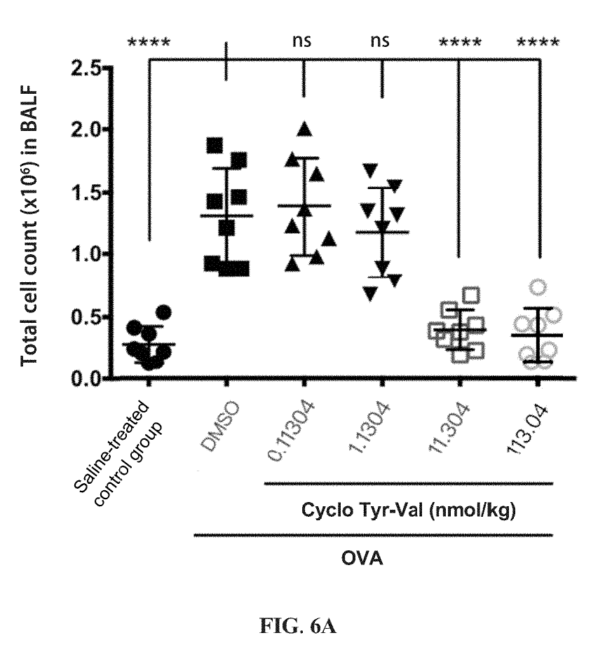

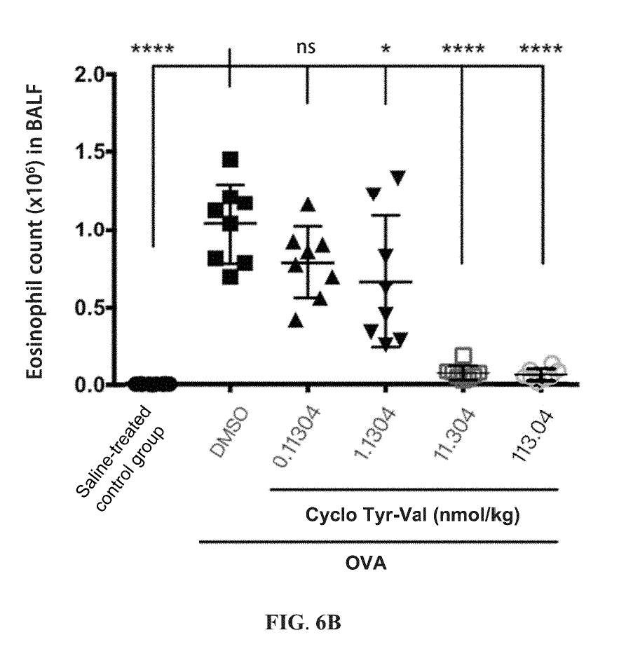

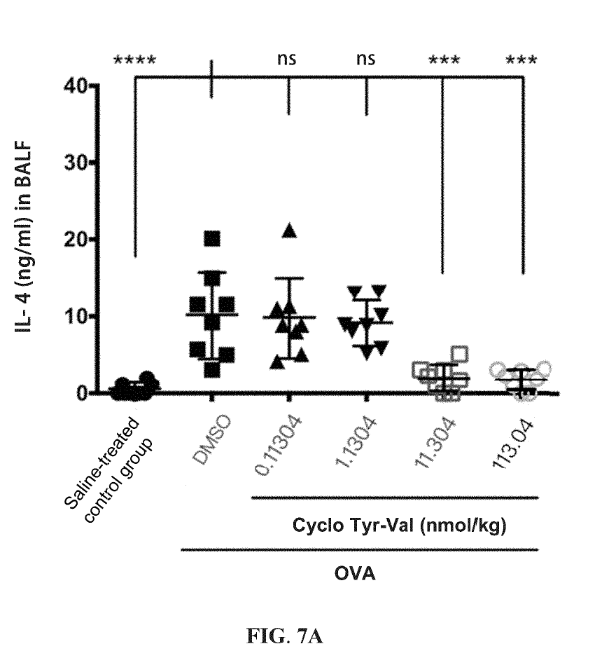

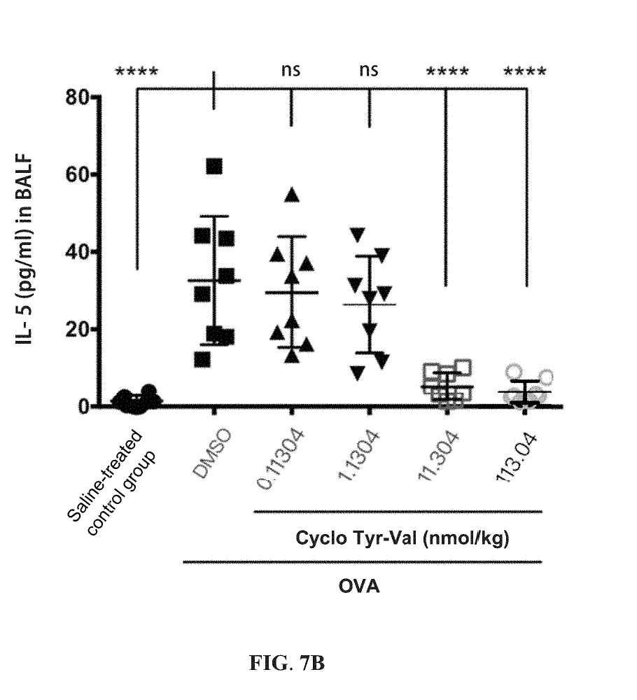

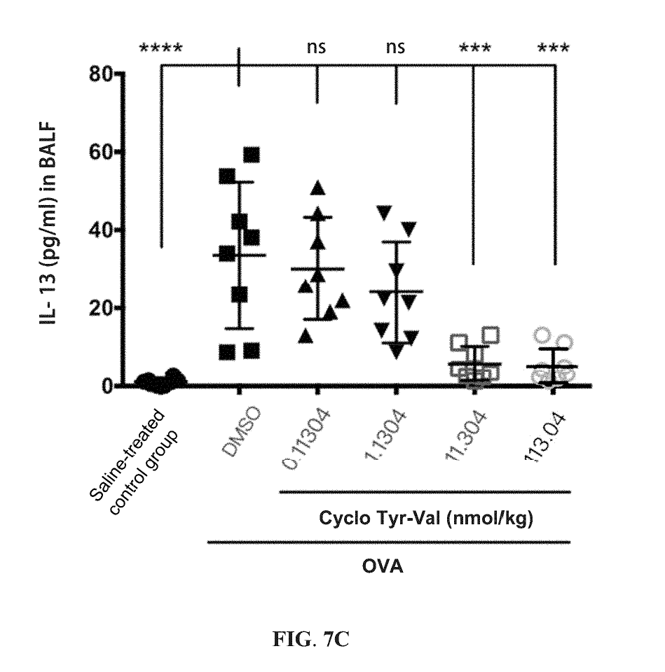

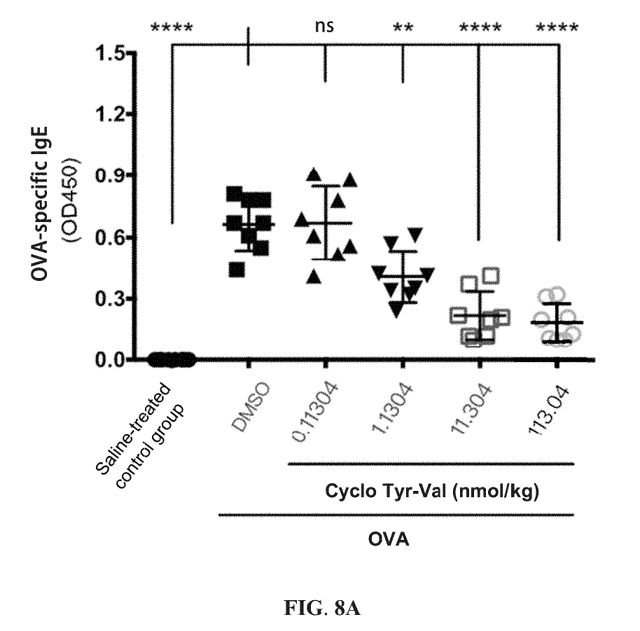

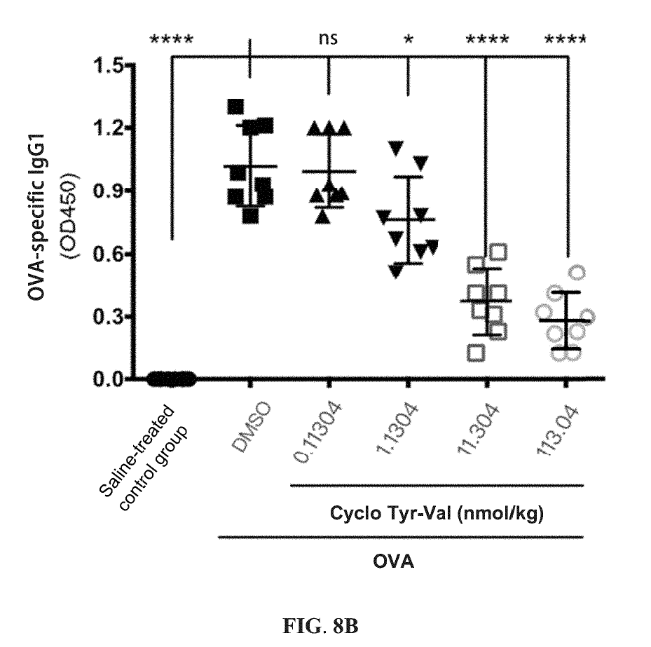

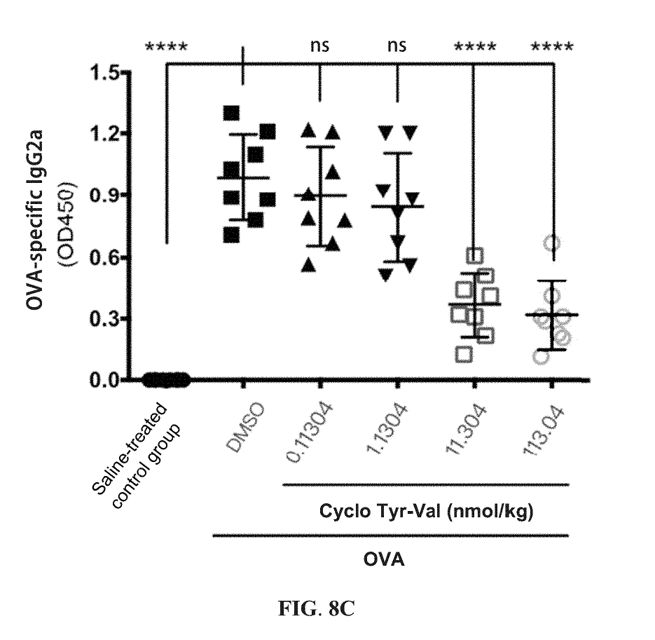

As shown in FIGS. 6A-6B, cyclo Tyr-Val reduced OVA-induced cell infiltration and eosinophilia in a dose-dependent manner in BALF. As shown in FIGS. 7A-7C, cyclo Tyr-Val also reduced OVA-induced secretion of IL-4, IL-5, and IL-13 in BALF. Consistent with these results, cyclo Tyr-Val reduced OVA-specific IgE and IgG production in a dose-dependent manner in serum (FIGS. 8A-8C).

In summary, the results of Examples 2 and 3 indicate that the symptoms of asthma and airway allergies can be alleviated by administering cyclo Tyr-Val during the development of OVA-induced asthma in mice. Therefore, cyclo Tyr-Val can prevent exacerbation of this disease or condition.

### Example 4

### Effects of the Mode of Administration of Cyclo Tyr-Val on its Anti-Asthmatic Activity

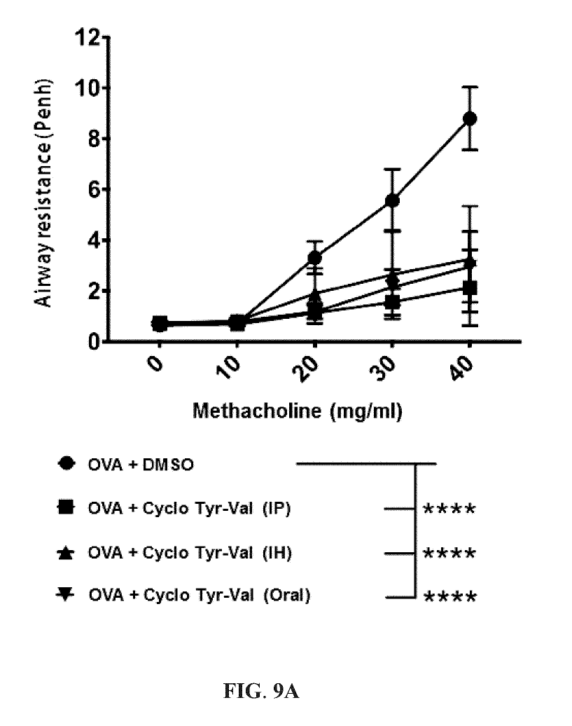

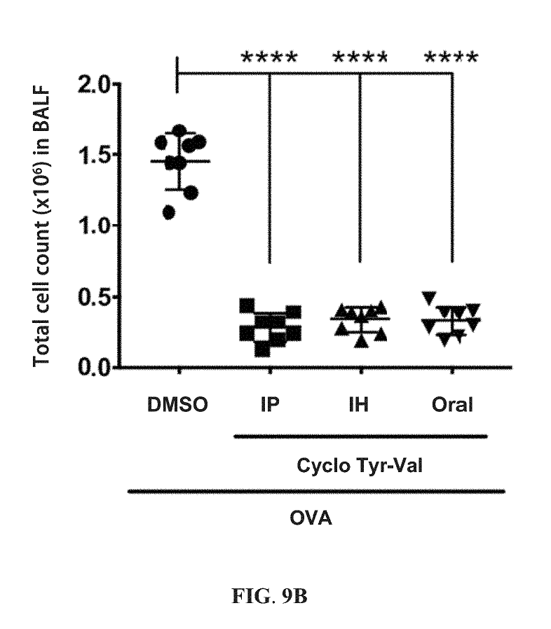

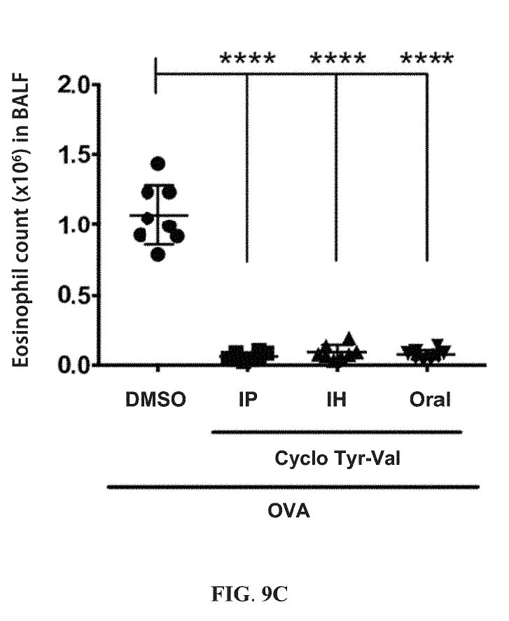

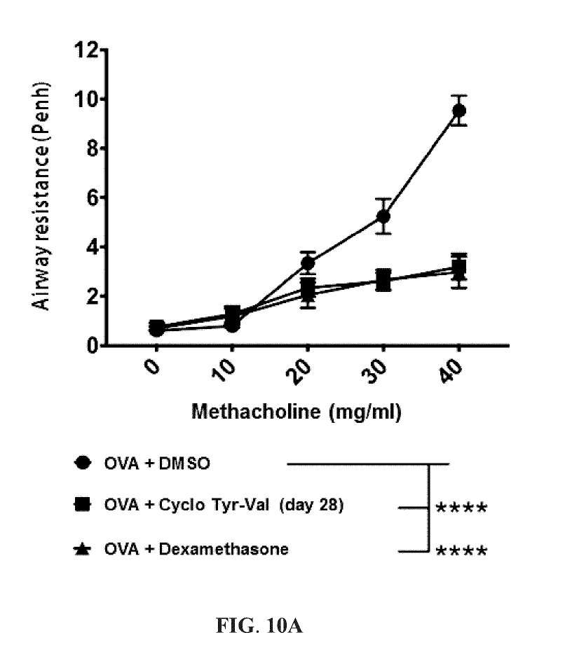

This example illustrates the effects of the mode of administration of cyclo Tyr-Val on asthmatic mice. OVA-induced asthmatic mice (BALB/c) were established according to the method described in Example 2. On day 21, the mice were treated intraperitoneally (IP), by inhalation (IH), or orally with cyclo Tyr-Val (at a dose of 11.304 nmol/kg of body weight per day) for 7 days, followed by assessment of AHR, total cell count in BALF, and eosinophil count in BALE OVA-induced asthmatic mice that were administered daily with the vehicle (0.5% DMSO in saline) intraperitoneally were used as control. As shown in FIGS. 9A-9C, treatment of OVA-induced asthmatic mice with cyclo Tyr-Val for 7 days via intraperitoneal injection, inhalation, or the oral route reduced AHR, total BALF cell count, and BALF eosinophil accumulation, compared with the vehicle-treated group.

### Example 5

### Comparison of the Anti-Asthmatic Effect of Cyclo Tyr-Val and Dexamethasone

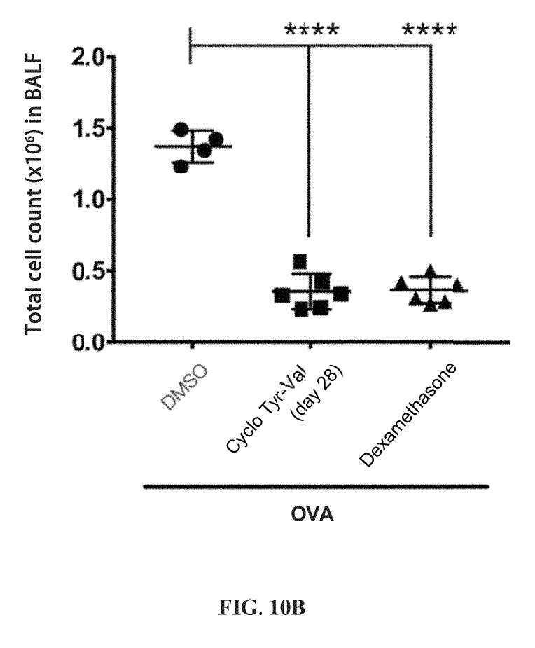

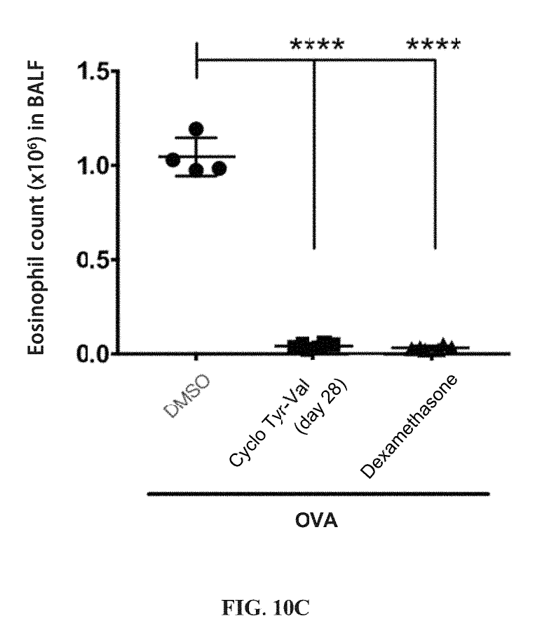

In this example, the anti-asthmatic effect of cyclo Tyr-Val was compared with those of dexamethasone, a pharmaceutical drug conventionally used to treat asthma. OVA-induced asthmatic mice (BALB/c) were established according to the method described in Example 2. The mice were treated with 2% OVA using a nebulizer on day 30 and 33 and were intraperitoneally treated with the vehicle (0.5% DMSO in saline) or cyclo Tyr-Val (at a dose of 11.304 nmol/kg of body weight per day) on day 28 to 34, followed by measurement of AHR, total cell count in BALF, and eosinophil count in BALF. Another group of OVA-induced asthmatic mice that were treated orally with dexamethasone (at a dose of 3 mg/kg of body weight per day) on day 28 to 34 were used as a positive control. As shown in FIGS. 10A-10C, treatment with cyclo Tyr-Val after establishment of OVA-induced AHR (day 28) in mice significantly reduced AHR, BALF total cell count, and BALF eosinophil accumulation, compared with the vehicle-treated group, and the inhibitory effects were comparable to those observed for dexamethasone. These results indicate that cyclo Tyr-Val may be used as an alternative to dexamethasone for treatment of asthma and airway allergies.

In one preferred embodiment, the effective amount of cyclo Tyr-Val or related cyclodipeptide compound that would produce anti-asthmatic effects in a human subject (with an average body weight of 70 kg) is estimated at 0.21 mg/individual per day, which is equal to 0.003 mg/kg of body weight or 11.304 nmol/kg of body weight.

### Example 6

### Effects of Cyclo Tyr-Val on Hepatic or Renal Functions in Mice

The effects of cyclo Tyr-Val treatment on hepatic and renal functions in mice were evaluated to determine if the compound produces toxic side effects. For these experiments, saline-treated mice (control group) or OVA-induced asthmatic mice were intraperitoneally treated with the vehicle (0.5% DMSO in saline) or cyclo Tyr-Val (at a dose of 11.304 nmol/kg), followed by measurement of blood biochemical parameters, including levels of aspartate transaminase (AST), alanine transaminase (ALT), total bilirubin (T-BIL), albumin (ALB), blood urea nitrogen (BUN), creatinine (CREA), and uric acid (UA). The blood biochemical parameters were monitored using a Hitachi 7080 biochemical analyzer (Hitachi, Japan). As shown in TABLE 1, treatment with cyclo Tyr-Val did not significantly affect hepatic or renal functions in the two groups of mice, suggesting that the compound does not produce toxic side effects.

TABLE 1
Blood biochemical parameters in mice

| | | | OVA-induced | |
|---|---|---|---|---|
| | Control mice | | asthmatic mice | |
| | | Cyclo | | Cyclo |
| | Vehicle | Tyr-Val | Vehicle | Tyr-Val |
| AST | 68.375 ± 6.085 | 70.775 ± 3.056 | 70.619 ± 2.605 | 69.718 ± 4.115 |
| (U/l) | | | | |
| ALT | 26.535 ± 4.173 | 26.283 ± 2.813 | 26.931 ± 2.731 | 26.673 ± 2.279 |
| (U/l) | | | | |
| T-BIL | 27.561 ± 4.516 | 26.169 ± 3.536 | 25.638 ± 2.640 | 26.508 ± 2.454 |
| (μg/dl) | | | | |
| ALB | 3.030 ± 0.308 | 2.669 ± 0.480 | 2.778 ± 0.331 | 2.737 ± 0.322 |
| (g/dl) | | | | |
| BUN | 31.145 ± 4.893 | 26.588 ± 2.756 | 26.796 ± 2.054 | 25.864 ± 2.638 |
| (mg/dl) | | | | |
| CREA | 0.285 ± 0.140 | 0.236 ± 0.124 | 0.268 ± 0.073 | 0.250 ± 0.078 |
| (mg/dl) | | | | |
| UA | 2.703 ± 0.642 | 2.979 ± 0.412 | 2.721 ± 0.314 | 2.690 ± 0.275 |
| (mg/dl) | | | | |

The data are based on duplicate experiments (n = 8 mice per group).

The experimental results mentioned above shows that cyclo tyrosine-valine significantly reduces the symptoms of asthma and airway allergies in mammals, while producing no toxic side effects. Therefore, the present invention provides a new strategy to treat asthma and airway allergies using cyclo tyrosine-valine or related cyclodipeptide compounds. In view of the pressing need in the market for therapies to treat these conditions, the present invention has obvious commercial value.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for treating asthma and airway allergy, comprising administering to a subject in need thereof an effective amount of cyclo tyrosine-valine or a related cyclodipeptide compound.

2. The method of claim 1, wherein the effective amount of the cyclo tyrosine-valine or the related cyclodipeptide compound is from 0.001 mg/kg to 1,000 mg/kg of body weight per day.

3. The method of claim 1, wherein the effective amount of the cyclo tyrosine-valine or the related cyclodipeptide compound is 0.003 mg/kg of body weight per day.

4. The method of claim 1, wherein the cyclo tyrosine-valine or the related cyclodipeptide compound is administered via intraperitoneal injection, inhalation, or by mouth.

5. The method of claim 1, wherein the related cyclodipeptide compound is cyclo tyrosine-leucine.

6. The method of claim 1, wherein the cyclo tyrosine-valine or the related cyclodipeptide compound reduces secretion of a pro-inflammatory cytokine.

7. The method of claim 6, wherein the pro-inflammatory cytokine is selected from the group consisting of interleukin-4, interleukin-5, interleukin-13, and any combinations thereof.

8. The method of claim 1, wherein the cyclo tyrosine-valine or the related cyclodipeptide compound inhibits airway hyperresponsiveness (AHR).

9. The method of claim 1, wherein the cyclo tyrosine-valine or the related cyclodipeptide compound reduces eosinophil accumulation in the lung.

10. The method of claim 1, wherein the cyclo tyrosine-valine or the related cyclodipeptide compound reduces production of an allergen-specific antibody.

11. The method of claim 10, wherein the allergen-specific antibody is an immunoglobulin E, an immunoglobulin G, or combinations thereof.
